(19) United States
(12) Patent Application Publication
(10) Pub. No.: US 2007/0203755 A1
(43) Pub. Date: Aug. 30, 2007

(54) Medication Administration Information and User Interface System

(75) Inventors: Diana F. Willson, Durango, CO (US); Lynne E. Valentine, Covington, LA (US); Hilari K. Jones, Salt Lake City, UT (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., MALVERN, PA (US)

(21) Appl. No.: 11/677,094

(22) Filed: Feb. 21, 2007

Related U.S. Application Data
(60) Provisional application No. 60/777,601, filed on Feb. 28, 2006.
(60) Provisional application No. 60/779,627, filed on Mar. 6, 2006.

(51) Int. Cl. A61B 5/00

(52) U.S. Cl. 705/3

(57) ABSTRACT

A system supports a nursing medication management workflow by providing both a work shift overview of a medication regimen giving nurses a comprehensive view of information needed to plan and track medication administrations throughout a shift and an intuitive and quick way for nurses to document medication administrations. A medication administration information system includes at least one repository of information identifying medications and past and future times for administering medications to patients. A display processor employs data retrieved from the at least one repository in initiating generation of data representing at least one display image presenting tabular data indicating medications previously administered, and to be administered, to a particular patient together with a time indication of administration, for the duration of a work shift associated with the particular patient.

This is a non-provisional application of provisional application Ser. No. 60/777,601 by D. F. Willson et al. filed Feb. 28, 2006 and of provisional application Ser. No. 60/779,627 by D. F. Willson et al. filed Mar. 6, 2006.

### FIELD OF THE INVENTION

This invention concerns a medication administration information system indicating medications previously administered, and to be administered, to a particular patient together with a time indication of administration.

### BACKGROUND OF THE INVENTION

In a hospital, when nurses start a new work shift, they need to learn about a medication regimen for each patient. Nurses need to quickly understand a medication schedule for a patient so that tasks can be planned for a day and so that scheduled medications are administered in a timely manner. In addition to scheduled medication administrations, a nurse needs to know when the next IV bag of a continuous infusion is due to be hung and to understand what “as needed” (PRN) medications can be given based on changing patient parameters throughout a work shift. This is a large amount of information that is difficult to assimilate in a short period of time and nurses sometimes resort to creating their own, paper schedule to try to manage the information. Hand-written paper schedules are difficult to read and fail to give a quick idea of a medication regimen throughout a shift. A paper schedule cannot notify a nurse about medications that are late, due now, or due in the future nor can they be automatically updated as new medications are added or discontinued. In addition, paper schedules fail to interactively support a nurse in documenting the administration of medications and they do not provide instantly readable information about these administrations.

Other known electronic Medication Administration Record (MAR) systems do not display a view of a medication regimen over a nurse work shift, for example. Instead, in known systems, medications are typically listed by date and time of the next medication administration. This requires a nurse to read through a chronological list of scheduled medication administrations and impedes obtaining a clear picture of a medication regimen within a work shift framework, for example. Although an interactive chronological listing of scheduled medications is minimally useful to a nurse, it does not support managing nursing workflow (a task sequence) or provide a cognitive road map from which to plan patient care.

Existing systems typically support pharmacy practice and/or mimic paper Medication Administration Records (MARs). Existing MAR systems do not provide a quick, cognitive road map indicating scheduled medications of a patient or information related to medications that have been administered throughout a work shift. A system according to invention principles addresses these deficiencies and related problems.

### SUMMARY OF THE INVENTION

A system supports a nursing medication management workflow by providing a work shift overview of a medication regimen giving nurses a comprehensive view of information needed to plan and track medication administrations throughout a shift as well as an intuitive and quick way for nurses to document medication administrations, using specific font styles and icons. A medication administration information system includes at least one repository of information identifying medications and past and future times for administering medications to patients. A display processor employs data retrieved from the at least one repository in initiating generation of data representing at least one display image presenting tabular data indicating medications previously administered, and to be administered, to a particular patient together with a time indication of administration, for the duration of a work shift associated with the particular patient.

### BRIEF DESCRIPTION OF THE DRAWINGS

FIG. 1 shows a medication administration information system, according to invention principles.

FIG. 2 shows a medication administration record structure (MAR Grid), according to invention principles.

FIG. 3 shows workflow functions associated with a medication administration record structure, according to invention principles.

FIG. 4 shows a user interface display image enabling a user to enter medication administration information, according to invention principles.

FIG. 5 shows a user interface display image enabling a user to view and edit entered medication administration information, according to invention principles.

FIG. 6 shows a user interface display image indicating a medication administration history, according to invention principles.

FIG. 7 shows a user interface display image enabling a user to document medication infusion information, according to invention principles.

FIG. 8 shows a user interface display image presenting information for multiple different types of tasks for multiple different patients in a work shift, according to invention principles.

FIGS. 9 and 10 illustrate a user interface display image indicating information for multiple different types of tasks for multiple different patients in a work shift in collapsed and expanded form, according to invention principles.

FIG. 11 shows a user interface display image indicating filtered medication administration information, according to invention principles.

FIG. 12 shows a table indicating icons displayable in the MAR Grid of FIG. 2 identifying medication administration information, according to invention principles.

FIG. 13 shows a flowchart of a process performed by a medication administration management and treatment planning system, according to invention principles.

### DETAILED DESCRIPTION OF THE INVENTION

A processor, as used herein, operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor comprise any combination of hardware, firmware, and/or software.

An executable application, as used herein, comprises code or machine readable instructions for conditioning the processor to implement predetermined functions, such as those of an operating system, a context acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A user interface (UI), as used herein, comprises one or more display images, generated by the display processor under the control of the processor enabling user interaction with a processor or other device and associated data acquisition and processing functions. The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of the executable procedure or executable application manipulates the UI display images in response to the signals received from the input devices. In this way, the user interacts with the display image using the input devices, enabling user interaction with the processor or other device. A workflow processor initiates and manages a sequence of tasks for performance by a worker, device or both such as providing a patient in a care setting with medication and treatment. The workflow processor provides data representing a task list of one or more workers and updates and manages the task list. A care setting comprises an inpatient setting or an outpatient setting and includes, for example, a patient room, a room in a particular hospital department, treatment station room such as a diagnostic imaging (e.g., MRI, CT scan, X-ray, Ultrasound) or therapeutic (e.g., radiation or sonic) therapy station or physical therapy room. The functions and process steps herein may be performed automatically or wholly or partially in response to user command. An activity performed automatically is performed in response to executable instruction or device operation without user direct initiation of the activity.

A system according to invention principles provides a Medication Administration Record (MAR) image display including a Grid data structure comprising an intuitive and interactive medication administration function that specifically supports nursing workflow within a framework of a specific nursing work shift, for example. The system uses special font styles and icons to provide a substantially real time, informational view of a selected patient medication regimen. A work shift view of a MAR includes the last two hours of a previous work shift, for example to help prevent common medication errors that occur during shift change. At the start of a shift, the nurse can see at a glance what medications are ordered for a patient, which medications are scheduled to be administered during a shift, and at what times. A nurse can also see if the patient has any continuously running infusions and, if so, the rate of infusion and the estimated time for a new infusion bag to be provided. Throughout a work shift, changing font styles, symbols or icons indicate which medications are late, due now, or still due to be administered in the future. As a nurse, for example, uses a MAR to document the administration of medications, the MAR is updated and special icons convey important information about the administration, such as whether the medication was fully administered, when the administration occurred, and whether it was administered early or late based on facility-defined rules. The system presents a shift-based framework that reflects a workflow task sequence and practice patterns nurses use to plan patient care and medication administrations and not merely a chronological list of scheduled medications used in known systems.

The system MAR Grid provides an interactive medication administration function that supports nursing workflow within the framework of a nursing shift. The MAR advantageously provides an intuitive visual representation of the process and status of medication administration throughout a work shift. Nurses can use the shift view to plan and chart the administration of medications throughout the shift.

The system uses dynamic font styles and icons within the MAR framework and accelerates providing information related to scheduled medications and those that have been administered during the shift. Thereby, at a glance, nurses can determine whether there are any immediate Stat, early, or late medication administrations, which medications are due now and in the future, what PRN (“as needed”) medications the patient is prescribed and when they were last given and whether the patient is on any continuous infusions. Further, if the patient is on any continuous infusions, nurses can determine the last recorded infusion volume and when the next infusion bag is estimated to be needed to be provided. Often several medications are scheduled to be administered at the same time to a patient. The system arranges nursing workflow to administer these medications to the patient during the same trip to the patient's bedside. From within the MAR Grid, nurses can quickly document multiple medication administrations on one display image while still entering required medication-specific information. The MAR Grid function facilitates recording of multiple medications scheduled and administered during a common time period. Nurses and other clinicians are able to use the MAR Grid to review the scheduled medication administrations for future shifts up to 24 hours in advance. In addition, the MAR Grid provides a user the ability to view medication administration status in previous work shifts, along with medications administered during those shifts.

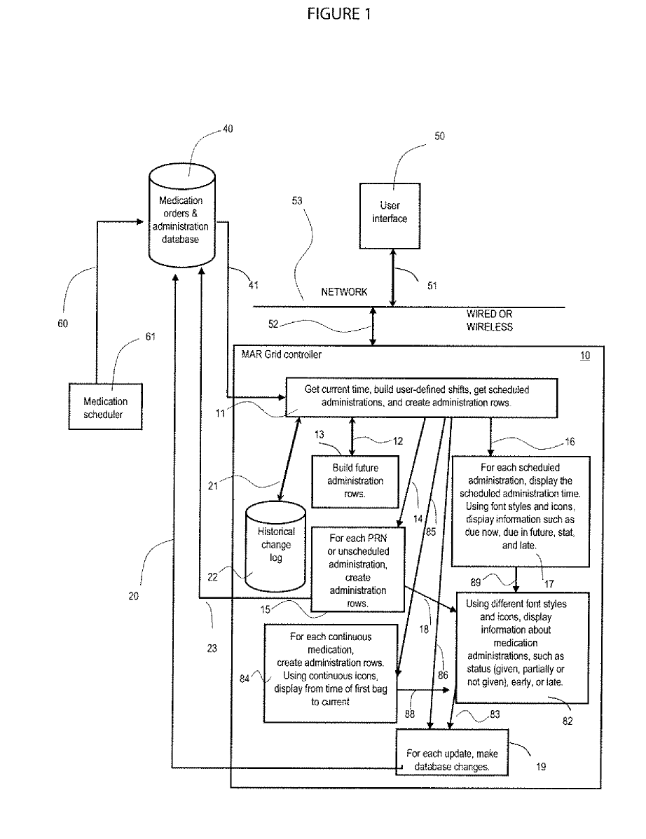

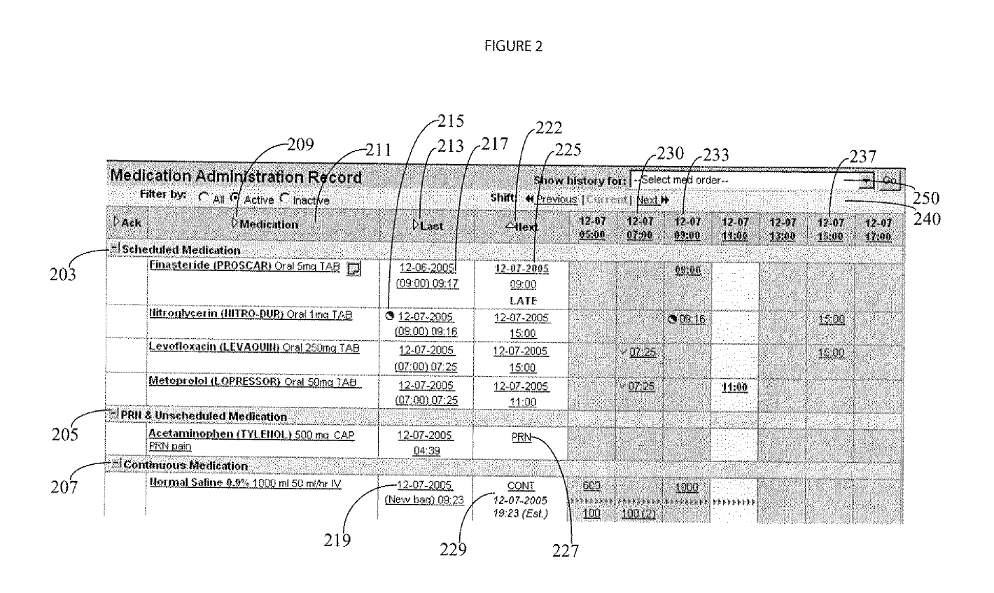

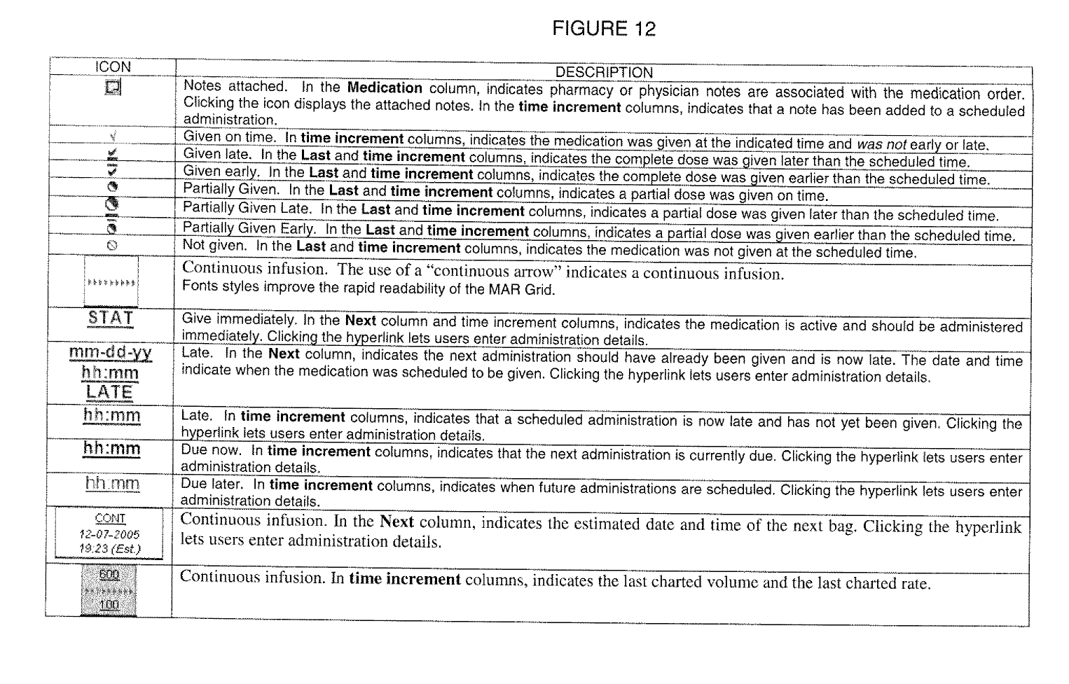

FIG. 1 shows a medication administration information system for providing a MAR Grid as illustrated in FIG. 2. Specifically, FIG. 2 shows a medication administration record structure (MAR Grid) generated by the system of FIG. 1. In one embodiment medications are organized into expandable and collapsible segments comprising categories including, for example, Scheduled medications 203, PRN and unscheduled medications 205, Continuous medications 207 and inactivated medications during a shift (not shown). Within a category, an individual image display row represents a specific medication order. An individual image display column contains data concerning a medication order in a corresponding row. Columns that are sortable are identified with an icon (e.g., 209) and Medication column 211 displays medication order hyperlinks that are selectable to display order details (e.g., identifying physician placing the order for medication to be administered to a patient, patient identifier, related notes etc.). Last column 213 displays a date and time of the last administration of a medication together with a corresponding scheduled administration time displayed in parentheses. Icons (e.g., icon 215) indicate if a medication was partially administered and/or if it was administered early or late, as defined by a facility such as a hospital. FIG. 12 shows a table illustrating additional icons displayable in the MAR Grid of FIG. 2 identifying further medication administration detailed information. In response to user selection of a date and time hyperlink (e.g., link 217, FIG. 2), the system displays a read-only view of medication administration details. In the case of a medication comprising a continuous infusion, Last column 213 displays a date and time (e.g., 219) the last infusion bag was hung.

Next column 222 displays a date and time of a next scheduled medication administration. The system automatically sorts the values in column 222 so that immediate Stat and Late administrations display at the top, followed by routine administrations in chronological order. The system further employs font style changes to provide additional information about Stat and Late administrations. In response to user selection of a date and time hyperlink in column 222 (e.g., hyperlink 225), a display image window enabling user entry of medication administration details is displayed. A key word, such as PRN 227 is displayed as a hyperlink for medications without scheduled administration times. In response to user selection of such a hyperlink (e.g., hyperlink 227), a display image window enabling user entry of medication administration details is displayed. Further, the system displays an estimated date and time 229 a next infusion bag is due to be hung for a continuous infusion.

The time increment columns, including columns 230 and 233, for example, display a current work shift in two-hour time increments as well as the last two hours of a previous work shift and any scheduled administrations as well as administration events. The shift length is determined by a unit where a patient is admitted. If the patient is not admitted to a unit, a 12-hour default work shift length is used. A MAR Grid dynamically adjusts to accommodate shifts that are 6, 8, or 12 hours long, for example. The times of scheduled medication administrations are displayed in an appropriate time increment column. In response to a user clicking a time hyperlink of a time of a scheduled medication administration, the system displays an image window enabling medication administration data entry. The actual times of medication administration are displayed in a time increment column for those scheduled administrations that have been completed. In response to a user clicking a time hyperlink of a completed administration, the system displays an image window indicating medication administration detailed information.

In response to user selection of a hyperlinked column header in a time increment, e.g. hyperlinked time header 237, the system displays data in a single display image indicating scheduled medication administrations that are currently due in the corresponding time increment. The MAR Grid further supports nurse documentation of the administration detail for one or more of the medications that are displayed. The system employs filters to determine the scope of medications displayed in the MAR Grid. The filter enables display of all, active or inactive medications for a user (e.g., a user system account) in response to corresponding user selectable options in row 240. Further, Current, Previous, and Next hyperlinks in row 240 enables a user to view a MAR Grid for different shifts. A Current shift is a default view. The Previous hyperlink enables a clinician to view previous shifts and associated medication administrations during that shift. The Next hyperlink enables a clinician to view scheduled administrations up to 24 hours in the future, for example.

A system security processor determines user authorization to view and edit medication administration data in a MAR as well as to enter data for incorporation in a MAR in response to security configuration setting information set by an administrator, user or other authorized party. Security configuration settings may determine that a particular user, or a user performing a particular care role, is able to have read-only or read-write access to MAR data or able to modify/delete data entered by another user within a user configurable period of time. Further, Show History option list 250 enables a user to select a particular medication order (active or inactive) and to initiate display of an image window. The image window shows the medication administrations performed for the selected particular medication order as well as information specific to that order, such as the various dosages (for range and sliding scale medication orders) or anatomical administration sites (for intramuscular or subcutaneous medication orders), for example. Show History option list 250 also provides a user selectable link to detailed information for the selected particular medication order and a selectable link to display images enabling a user to enter data for medication administrations yet to be performed.

FIG. 1 shows a medication administration information system for providing a MAR Grid as illustrated in FIG. 2. A user interacts with MAR Grid Controller 10 via user interface 50 initiating bidirectional communication through network 53 and communication links 51 and 52. Medication scheduler 61 employs an independently executed procedure that is activated on a user defined schedule to produce future (prospective) medication administration records in Medication Orders & Administration database 40 via communication path 60. Medication scheduler 61 produces administration records for a user defined period of time occurring in the future. MAR Grid Controller 10 in step 11 acquires a current time, generates data representing user defined work shifts and acquires data indicating medication administrations scheduled for a patient for incorporation in rows for display by user interface 50. MAR Grid Controller 10 in step 13 via link 12 generates data representing image rows including prospective medication administration information for display in a MAR Grid. Controller 10 in step 11 acquires information from Medication Orders & Administration database 40 through interface 41 for incorporation in MAR Grid rows for display by user interface 50.

For each PRN or unscheduled administration, MAR Grid Controller 10 in step 15 executes a PRN/unscheduled administration procedure using interface 14 to create MAR Grid rows for PRN and unscheduled $medication administrations. The created PRN and unscheduled medication administrations rows are processed by a display procedure in step 82 via program interface 18, stored in database 40 via interface 23 and displayed on user interface 50. Also, for each continuous medication, MAR Grid Controller 10 executes a continuous medication procedure in step 84 via program interface 85 to create MAR Grid medication administration rows for individual continuous medications. In response to completion of step 84, data representing the created MAR Grid medication administration rows is sent to the display procedure executed in step 82 via program interface 88. Further, for individual scheduled administrations, MAR Grid Controller 10 executes a Scheduled Administration procedure in step 17 via program. interface 16 to create and display administration data on user interface 50 including scheduled medication administration time and associated data (using font styles and icons) including, due now, due in future, Stat, and late. In response to creation of administration data and completion of step 17, the administration data is also provided to the display procedure executed in step 82 via program interface 89. The display procedure executed in step 82 uses different font styles and icons to display information associated with medication administration, such as status (given, partially or not given) and timing (early or late). In response to completion of the display procedure executed in step 82, individual rows of the MAR Grid are scanned for changes and individual changes are identified by an update database procedure in step 19 that is initiated via program interfaces 83 and 86. The update database procedure executed in step 19 processes data representing MAR Grid row data to update MAR Grid row data in medication and orders administration database 40 using program interface 20. MAR Grid Controller 10 also keeps track of changes, modifications and deletions to MAR Grid data in a historical change log in step 22 though interface 21. MAR Grid Controller 10 stores data identifying, changes to MAR Grid data, a user making changes, a source computer employed in making changes and time and date of changes.

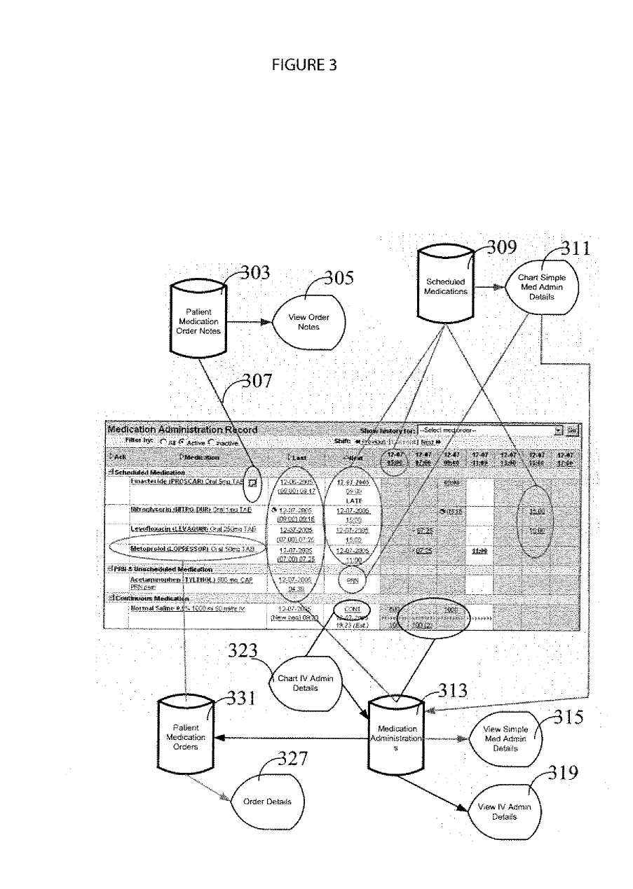

FIG. 3 shows workflow functions associated with a medication administration record structure. When medication notes are available for an order in step 303, the user is able to identify that the medication order notes are available from the associated displayed icon (indicated by path 307) and in step 305 initiates display of an image window including the notes by clicking the icon. In step 309, a display image window enabling user entry of medication administration details is displayed in response to user selection of a date and time hyperlink in the Next column or a time and or date in a time increment column. Similarly, in step 311 in response to user selection of a hyperlink for medications without scheduled administration times (e.g., a PRN hyperlink), a display image window enabling user entry of medication administration details is displayed. In response to completion of medication administration in step 313, a user is able to enter medication administration details for a continuously administered medication (e.g, an infusion) in step 323 via an image window displayed in response to clicking the word “CONT” in the Next column. A user accesses and views detailed information concerning an administered medication event in response to user selection of a date and time hyperlink in the Last column. A user further accesses and views detailed information concerning a simple medication administration and an infusion in steps 315 and 319 by selection of links in the MAR Grid. In steps 331 and 327 a user is able to select a medication order hyperlink and view detailed information concerning the selected order.

The MAR Grid indicates work shifts including the number of hours and the start time for specific care units. MAR Grid controller 10 uses this information to customize a work shift display for a particular care unit where a patient is admitted. Further, controller 10 uses special font styles and icons to facilitate user identification of medication status. A displayed work shift view as exemplified in FIG. 2 is usable as a clinician Worklist or Care Plan or to indicate a treatment Protocol and/or Care Path for a patient, for example. The MAR Grid treatment order structure is tailored for a care application to present ordered tasks in the MAR Grid with one task or order per row. A variety of different filters and sort functions are used to customize the display for different specialties, disciplines, and individual clinicians. The MAR Grid facilitates the planning and recording of medication administration information throughout a work shift and its shift-based structure is readily adapted for more widespread clinical use as a framework for a Worklist or Care Plan, for example. In another embodiment, a display provides the Next column instead of shift-specific time columns and the Next column shows a chronologically arranged indication when the next medications are due.

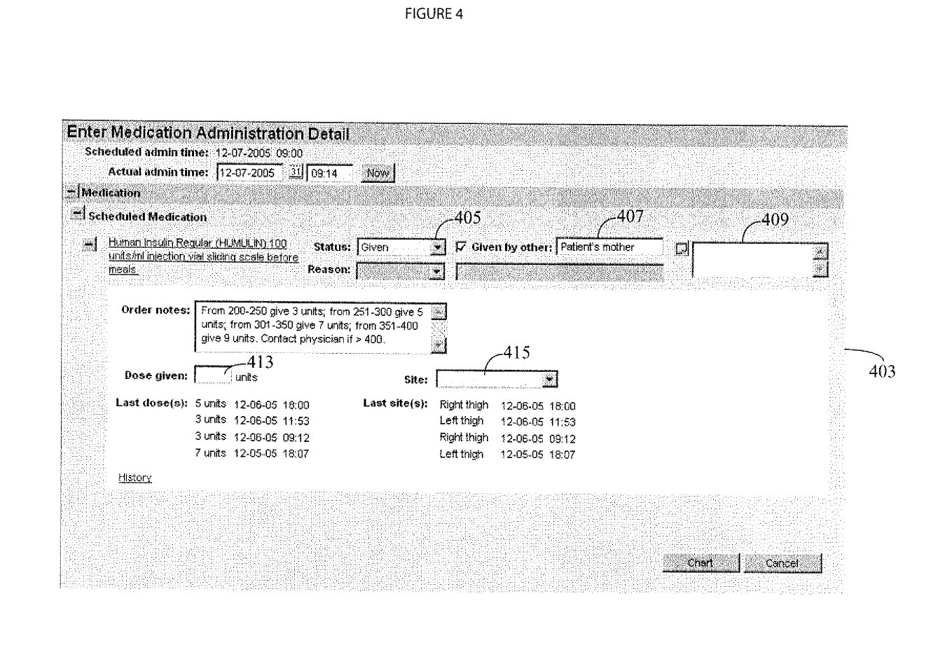

FIG. 4 shows user interface display image window 403 enabling a user to enter medication administration information. Display image window 403 is presented in response to user selection of Hyperlinked dates and times (or certain words for medications without scheduled administration times) in the MAR Grid (FIG. 2). Image window 403 enables a nurse, for example, to document medication administration status 405 (given, partially given, or not given), whether the medication was given by another person, and if so, who 407, free-text comments associated with the administration 409 and dose 413 and site 415 of administration, if required for a specific medication. Image window 403 also enables documentation of patient response for PRN medications and additional required data elements specific to a medication.

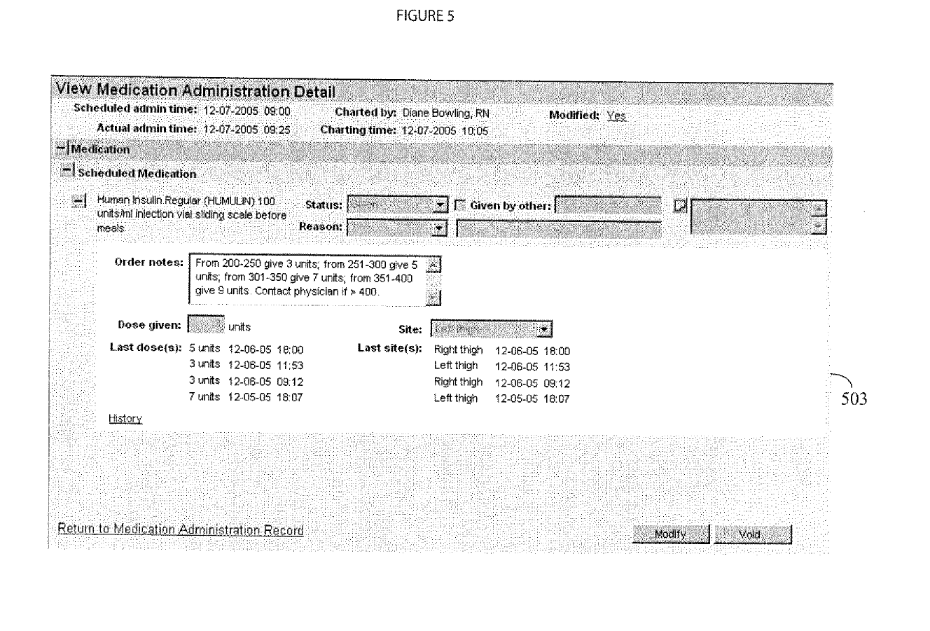

FIG. 5 shows a user interface display image window 503 enabling a user to view and edit entered medication administration information. Display of image window 503 is initiated in response to user selection of a hyperlinked date and time and associated symbol indicating that a medication has been administered in the MAR Grid (FIG. 2). Image window 503 enables a user to view previously documented data and modify or void the record.

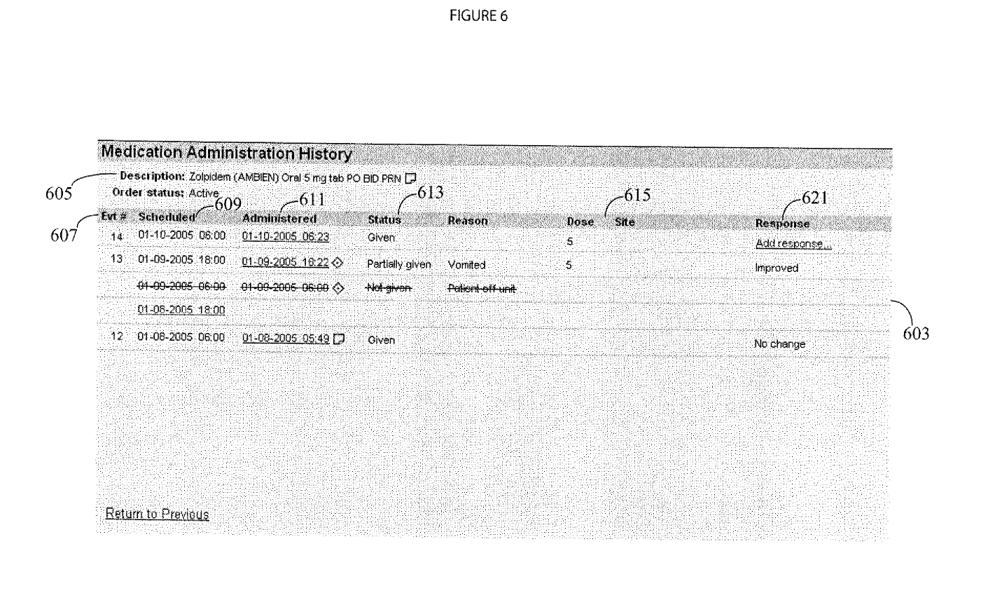

FIG. 6 shows user interface display image 603 indicating a medication administration history. Display image window 603 is presented in response to user selection of image windows via the MAR Grid supporting user entry of medication administration information, viewing of medication administration information, viewing medication order information and the “Show history” dropdown list (item 250 on the MAR Grid of FIG. 2). Medication administration history display image 603 indicates administration information for selected medication 605, listed in reverse chronological order. Administration event number 607 corresponds to the number of times the medication was administered. Display image 603 further shows scheduled administration date and time 609, actual administration date and time 611, status of medication administration (given, partially given, not given) 613 and other data specific to a medication, such as dose and site of administration 615. Display image 603 also includes data indicating patient response for PRN medications and other data collected at the time of administration, such as glucose level.

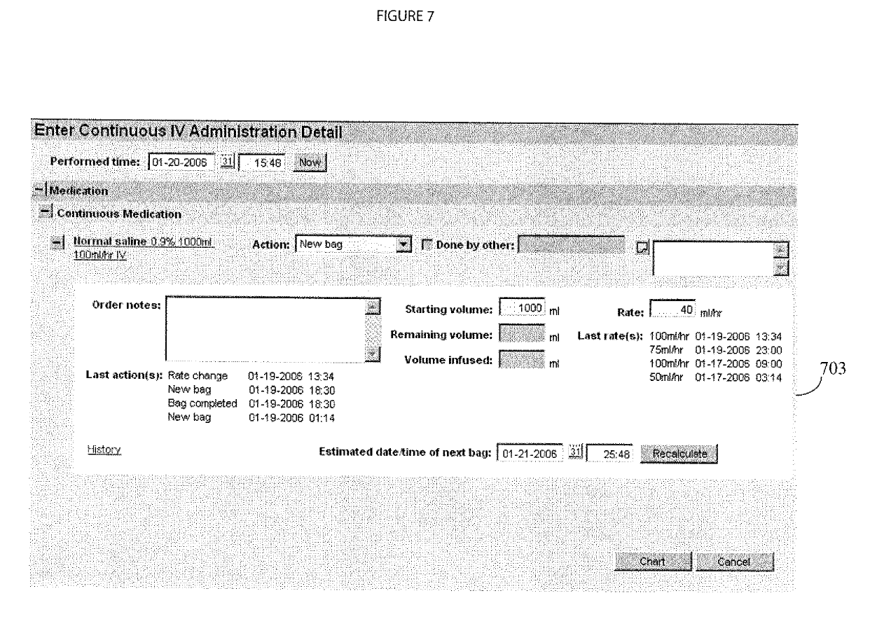

FIG. 7 shows user interface display image 703 enabling a user to document medication infusion information. Display image window 703 is presented in response to user selection of a “CONT” hyperlink in the MAR Grid of FIG. 2. Display image window 703 displays the entry screen where users can document actions such as provision of a new infusion bag, rate, bag completed, and fluid intake totals.

System embodiments encompass different configurations of MAR Grid. A clinician-based MAR Grid (as opposed to patient-based grid) supports individual clinicians by providing a task list specific to an individual clinician, not to patients. This Grid addresses the fact that nurses and other clinicians are given tasks to do that are not patient-based. For example, nurses are assigned to test equipment, to attend in-services and training and nurses assigned to a night shift are assigned to do “chart checks”. In another embodiment a role-based MAR Grid displays tasks assigned to a role performed for one or multiple patients. In clinical settings in which a team manages patient care tasks, the MAR Grid supports a task list for the team or an individual role. This accommodates the case where care managers switch duties, for example.

Different configurations of MAR Grid support multiple patient MAR data, workflow management and different work shifts. In one embodiment a MAR Grid, instead of 2-hour time increment columns, employs 3-hour, 4-hour or other increments to support a workflow. This supports clinical settings that do not plan patient care activities within hourly shifts, but rather in days, weeks or monthly time increments such as a long term care facility preferring patient tasks organized by days or weeks. It also supports charting and documentation requirements incorporated into a weekly MAR Grid, for example. In another configuration, a MAR Grid presents medication information for multiple different patients. This supports a clinician who manages the care of multiple patients such as a nurse assigned five patients. The nurse uses the MAR Grid to plan an entire work shift facilitated by a view of medication administrations and other care tasks that need to be done for the assigned patients within a work shift.

In a worklist configuration, a MAR Grid shows patient-related tasks together in one grid and the system provides various filters to sort and refine displayed medication administration data so that administrations concerning an individual medication are displayed, for example. A nurse may have multiple patient related tasks to accomplish during a work shift in addition to a medication administration task. A nurse needs to follow orders or protocols that may prescribe a time for performing patient physical assessments, recording vital signs, recording fluid intake and output volumes, changing dressings and providing interventions. Further, some tasks have specified dates and times for when tasks need to be done and other tasks do not. However, these tasks may still be displayed and documented within the MAR Grid, in a similar manner to PRN or Unscheduled medications. The system also acquires medication administration related data for incorporation in a MAR Grid using bar-coding, RFID and other automated wireless, data acquisition systems to support medication administration and to better support nursing workflow. A nurse is able see an entire work shift of planned medication administration tasks in a MAR Grid and employ bar-coding systems, for example to support medication administration, documentation and nursing workflow. A MAR Grid supports Care Plans, Protocols, Care Paths and Care Process Models (a Care Plan (or equivalent) comprises lists of treatment orders, interventions, reminders, outcomes and other tasks that facilitate optimal patient care over a period of time). A MAR Grid in another configuration includes additional categories differentiating identified patient problems so that an entire patient plan with associated tasks is viewable at one time in a single user friendly image.

A MAR Grid in other embodiments advantageously provides Care Plans and Care Maps specific to a patient (not a particular worker) that may or may not include medications. A Care Plan may includes different sorts of tasks that need to be done for the patient (like laboratory tests, imaging (e.g., X-rays), medications, dressing changes, patient teaching, outcome evaluations, assessments). These tasks are specific to a patient and may be scheduled or unscheduled but are to be performed within a set time period. Different sorts of clinicians are able to use a MAR Grid to view a Care Plan to see tasks completed and tasks needing to be performed and what a clinician specifically needs to do during a work shift or other time increment. The system enables a user to document a task via the MAR Grid when the task is completed and the MAR Grid provides comprehensive user interactive functions.

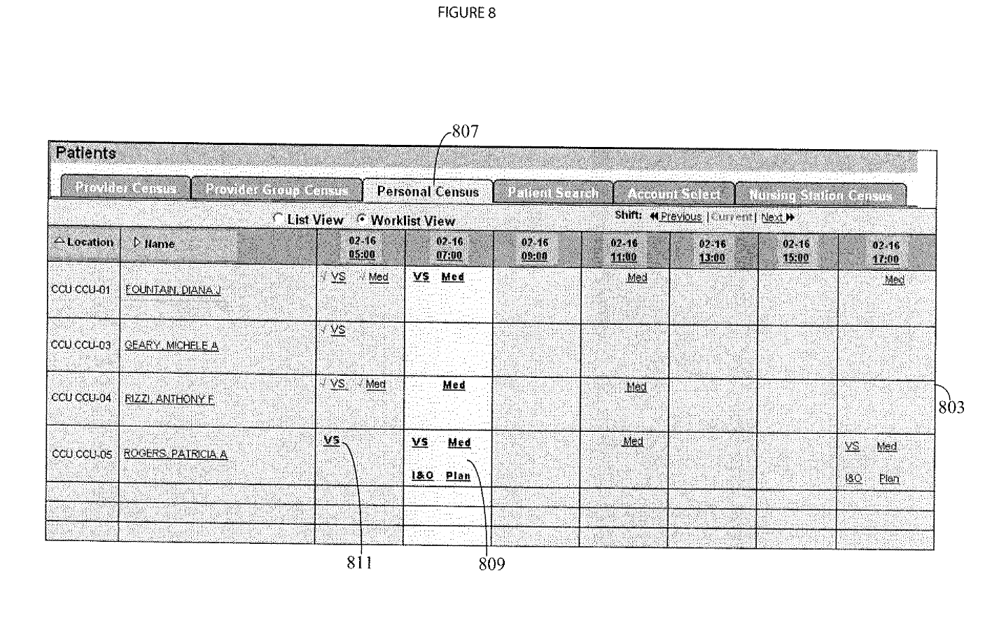

FIG. 8 shows user interface display image 803 presenting information for multiple different types of tasks for multiple different patients in a work shift. Display image 803 comprises a personal census 807 grid. with individual cells in a time increment column being divided into a quadrant. An upper left quadrant indicates Vital Signs orders, an upper right quadrant indicates Medication orders, a lower left quadrant indicates I&O (fluid input and output) orders, and a lower right quadrant indicates items on a care plan (e.g., a physical assessment) as illustrated in time increment cell 809. The system enables a user to configure the kinds of orders that make up a quadrant. As orders in an individual quadrant become due to be performed, display image 803 employs dark blue to indicate orders due to be performed now, light blue to indicate orders due to be performed in the future, a green check symbol to indicate orders were performed, bold red indicates orders are late. This color arrangement is used in a similar manner to the use of fonts and icons in a MAR Grid. Thereby, a nurse viewing image 803 sees red VS (vital signs) element 811 indicating that Patricia Rogers is late for having Vital Signs recorded. Other visual attributes may alternatively be used instead of, or in conjunction with, color. Similarly, the nurse sees that additional vital signs are now due on patients Fountain and Rogers, medication administrations are due for Fountain, Rogers and Rizzi, I&O fluid tasks are due for Rogers, and the Care Plan has an outstanding order for Rogers. In response to user selection of a Medication hyperlink the associated MAR Grid is presented. Similarly, user selection of a VS hyperlink initiates presentation of a VS flowsheet, user selection of an I&O hyperlink initiates presentation an I&O flowsheet, or user selection of a Plan hyperlink initiates presentation of a Care Plan.

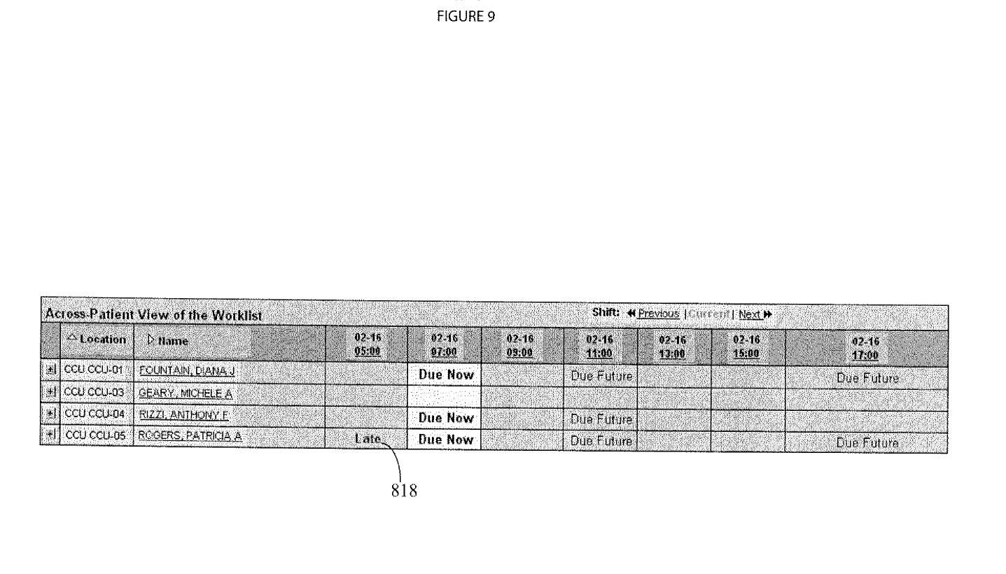

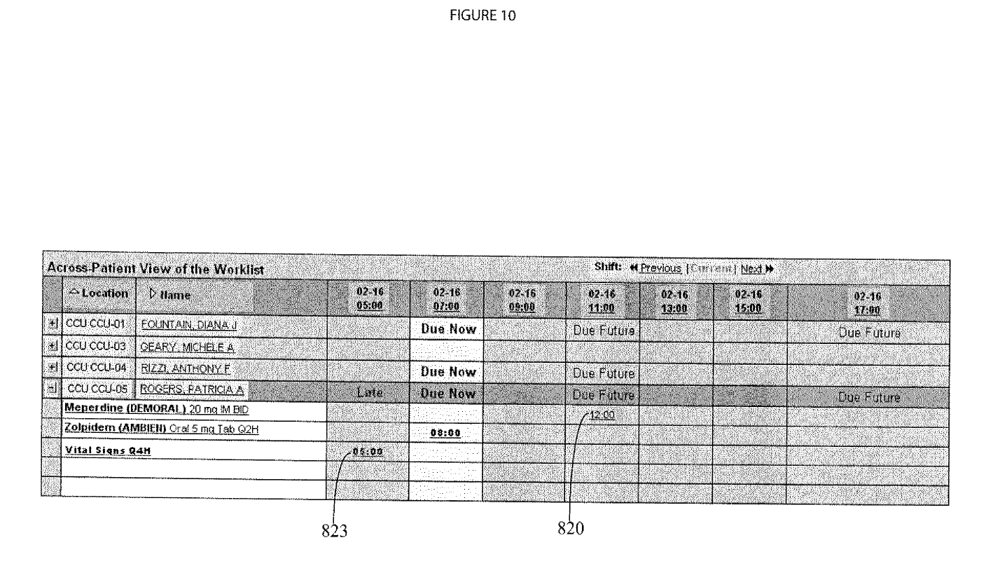

FIGS. 9 and 10 illustrate a user interface display image indicating information for multiple different types of tasks for multiple different patients in a work shift in collapsed and expanded form. FIG. 9 illustrates the information in a collapsed view which enables a user (e.g., a nurse) to see that Rogers has a late order 818 of some kind and that three patients have orders due now and the nurse can see when orders are due in the future. FIG. 10 indicates expansion of FIG. 9 for patient Rogers identifying multiple different types of tasks to be performed for patient Rogers in a work shift and enabling a nurse to initiate generation of an image window enabling task performance documentation. The different tasks include orders for medications and vital signs. In response to user selection of medication hyperlink 820 the user is presented with a medication administration data entry and capture image window specific to the medication concerned. In response to user selection of vital sign hyperlink 823 the user is presented with a vital sign data entry and capture image window.

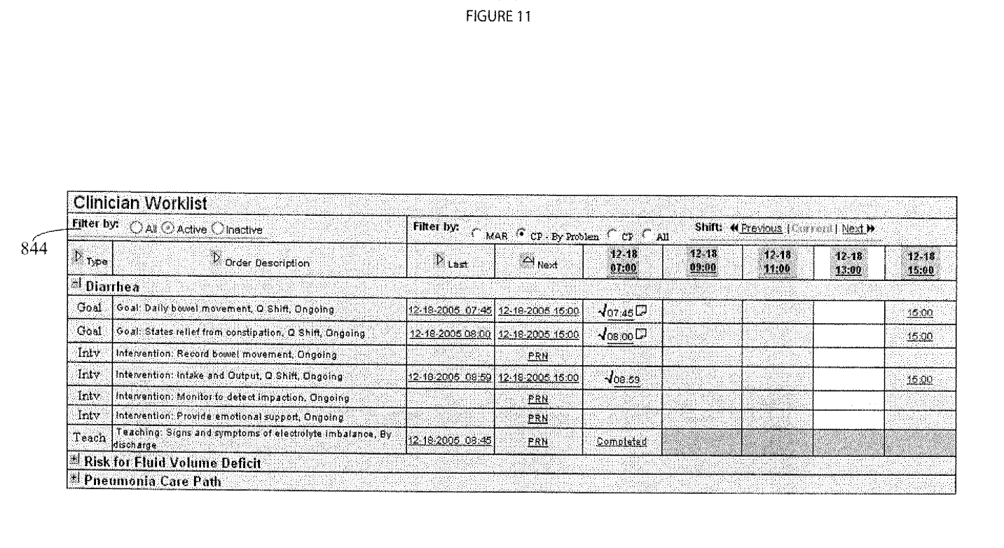

FIG. 11 shows a user interface display image indicating filtered work task information. The system enables a user, in response to item selection in row 844, to filter task information to present, Medication orders, tasks ordered by Care Plan (CP) diagnosed problem, tasks ordered by Care Plan (CP) and not grouped by diagnosed problem, All tasks and ordered medications from a MAR and Care Plan (no groupings). The system further enables a user, in response to item selection in row 844, to present All, Active and Inactive medications and tasks. Clinicians are able to record performance of orders and, as in medication administration documentation, are able to document observation details (via links within the image window).

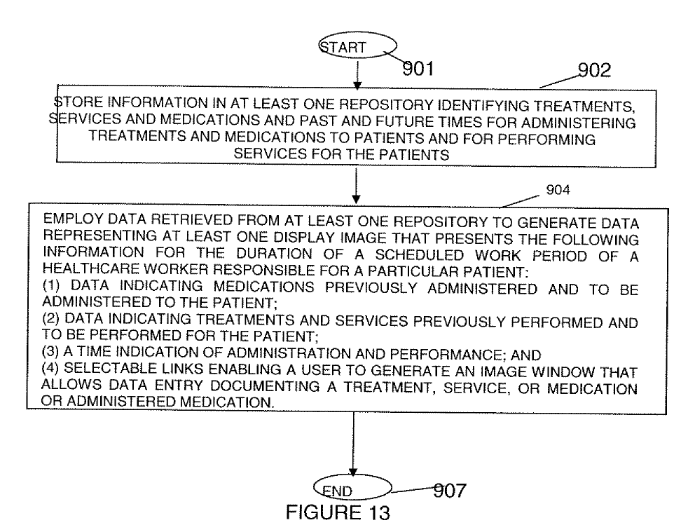

FIG. 13 shows a flowchart of a process performed by a patient fluid parameter user interface and processing system. In step 902 following the start at step 901 grid controller 10 stores in at least one repository, information identifying treatments, services and medications and past and future times for administering treatments and medications to patients and past and future times for performing services for the patients. The treatments and services comprise one or more of, obtaining and recording fluid volumes, a task identified in a care plan and obtaining patient fluid intake or output data.

In step 904 a display processor in controller 10 employs data retrieved from the at least one repository in initiating generation of data representing at least one display image. The at least one display image presents, data (tabular data in one embodiment) indicating treatments, services and medications previously administered and to be administered, to a particular patient and previously performed and to be performed, for a particular patient, together with a time indication of administration and performance. The at least one display image includes selectable links enabling a user to initiate generation of an image window enabling a user to enter data documenting a treatment, service or medication administered, for the duration of a scheduled work period of a healthcare worker responsible for healthcare for the particular patient. The at least one display image presents indications comprising data indicating at least one of, (a) a treatment and medication have been completely administered and a service has been completely performed, (b) a treatment and medication have not been administered and a service has not been performed, (c) a treatment and medication have been partially administered and a service has been partially performed and (d) a treatment and medication have been administered early or late and a service has been performed early or late, together with a visual attribute used to facilitate identification of individual indications. The visual attribute comprises color, highlighting, a symbol, shape, shading or text, for example.

The at least one display image presents, data (e.g., tabular data) indicating treatments, services and medications previously administered and to be administered, to multiple different patients and previously performed and to be performed, for multiple different patients, together with a time of a next medication or service administration and identifying a time a last medication or service was administered, for the duration of a work shift associated with a particular patient or multiple different patients. The at least one display image presents data enabling a user to select that the work shift is associated with a particular healthcare worker and/or associated with a particular healthcare worker role and also presents data enabling a user to adaptively select the work shift from between a work shift associated with a particular healthcare worker and a work shift associated with a particular healthcare worker role. The at least one display image presents data enabling a user to adaptively select a worker role from multiple different roles and the selected role has the work shift associated with a particular patient. Also in other configurations, the at least one display image presents data indicating different treatment related activities, exclusive of medication administration activity, performed by the particular worker, non-treatment related activities performed by the particular worker and data identifying the particular worker. The at least one repository of information in one embodiment identifies outcomes enabling evaluating outcomes of patients and the display image (or window in an image) enables a user to enter data documenting an outcome evaluation. The process of FIG. 13 terminates at step 907.

The system, processes and image displays of FIGS. 1-13 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A user interface according to invention principles may be used to present any kind of task list within a time framework. The user interface may be advantageously used to schedule time-critical tasks within predefined blocks of time (e.g., for farming, manufacturing, service-related, or other industries). Users of a MAR Grid, for example, may plan their time and document important information related to scheduled tasks. MAR Grid time increment columns may represent days, weeks, or months, for example, to support various planned tasks for any industry. A MAR Grid is also usable for other non-clinical purposes, including, for example, shops and any place that has employees may use it so that employees have a list of tasks and assignments to accomplish during an assigned shift. Any individual or employer may use it to manage assigned or planned tasks. Further, any of the functions and steps provided in FIGS. 1-13 may be implemented in hardware, software or a combination of both and may reside on one or more processing devices located at any location of a network linking the FIG. 1 elements or another linked network including another intra-net or the Internet.

What is claimed is:

1. A medication administration information system, comprising:
at least one repository of information identifying medications and past and future times for administering medications to patients; and
a display processor for employing data retrieved from said at least one repository in initiating generation of data representing at least one display image presenting tabular data indicating medications previously administered, and to be administered, to a particular patient together with a time indication of administration, for the duration of a work shift comprising a scheduled work period of a healthcare worker responsible for healthcare for said particular patient.

2. A system according to claim 1, wherein
said at least one display image presents data enabling a user to select said work shift is at least one of, (a) associated with a particular healthcare worker and (b) associated with a particular healthcare worker role.

3. A system according to claim 1, wherein
said at least one display image presents data enabling a user to adaptively select said work shift from between a work shift associated with a particular healthcare worker and a work shift associated with a particular healthcare worker role.

4. A system according to claim 1, wherein
said at least one display image presents indications comprising data indicating at least one of, (a) a medication has been completely administered, (b) a medication has not been administered, (c) a medication has been partially administered and (d) a medication has been administered early or late, together with a visual attribute used to facilitate identification of individual indications.

5. A system according to claim 4, wherein
said visual attribute comprises at least one of, (a) color, (b) highlighting, (c) a symbol, (d) shape, (e) shading and (f) text.

6. A system according to claim 1, wherein
said at least one display image presents data enabling a user to adaptively select a worker role from a plurality of different roles, said selected role having said work shift associated with said particular patient.

7. A system according to claim 1, wherein
said work shift associated with said particular patient is associated with a particular worker and
said at least one display image presents data indicating different treatment related activities, exclusive of medication administration activity, performed by said particular worker.

8. A system according to claim 1, wherein
said at least one display image presents tabular data indicating medications previously administered, and to be administered, to a plurality of different patients together with time indications of administration, for the duration of work shifts associated with said plurality of different patients.

9. A system according to claim 1, wherein
said work shift associated with said particular patient is associated with a particular worker and
said at least one display image presents data indicating different non-treatment related activities performed by said particular worker.

10. A system according to claim 1, wherein
said at least one display image presents tabular data indicating said medications previously administered, and to be administered, to a particular patient, together with data identifying said medications.

11. A system according to claim 10, wherein
said at least one display image presents tabular data indicating said medications previously administered, and to be administered, to a particular patient, together with data identifying a time of a next medication to be administered.

12. A system according to claim 10, wherein
said at least one display image presents tabular data indicating said medications previously administered, and to be administered, to a particular patient, together with data identifying a time a last medication was administered.

13. A system according to claim 10, wherein
said work shift associated with said particular patient is associated with a particular worker and
said at least one display image presents said tabular, together with data identifying said particular worker.

14. A treatment planning information system, comprising:
at least one repository of information identifying treatments, services and medications and past and future times for administering treatments and medications to patients and past and future times for performing services for said patients; and
a display processor for employing data retrieved from said at least one repository in initiating generation of data representing at least one display image presenting,
tabular data indicating treatments, services and medications previously administered and to be administered, to a particular patient and previously performed and to be performed, for a particular patient, together with a time indication of administration and performance, for the duration of a scheduled work period of a healthcare worker responsible for healthcare for said particular patient.

15. A system according to claim 14, wherein
said at least one display image presents indications comprising data indicating at least one of, (a) a treatment and medication have been completely administered and a service has been completely performed, (b) a treatment and medication have not been administered and a service has not been performed, (c) a treatment and medication have been partially administered and a service has been partially performed and (d) a treatment and medication have been administered early or late and a service has been performed early or late, together with a visual attribute used to facilitate identification of individual indications.

16. A system according to claim 14, wherein
said tabular data indicates treatments, services and medications previously administered and to be administered, to a plurality of different patients and previously performed and to be performed, for a plurality of different patients, together with a time indication of administration and performance, for the duration of a work shift associated with said particular patient.

17. A treatment work task management system, comprising:
at least one repository of information identifying treatments, services, outcomes and medications and past and future times for administering treatments and medications to patients and times for performing services and evaluating outcomes for said patients; and
a display processor for employing data retrieved from said at least one repository in initiating generation of data representing at least one display image presenting,
data indicating treatments, services and medications previously administered and to be administered, to a particular patient and previously performed and to be performed, for a particular patient, together with a time indication of administration and performance and selectable links enabling a user to initiate generation of an image window enabling a user to enter data documenting a treatment, service, outcome evaluation or medication administered, for the duration of a scheduled work period of a healthcare worker responsible for healthcare for said particular patient.

18. A system according to claim 17, wherein
said treatments and services comprise at least two of, (a) obtaining vital sign data, (b) a task identified in a care plan and (c) obtaining patient fluid intake or output data.

19. A system according to claim 17, wherein
said at least one repository of information identifies outcomes enabling evaluating outcomes for said patients and
said image window enables a user to enter data documenting an outcome evaluation.
